(19) United States
(12) Patent Application Publication
(10) Pub. No.: US 2024/0066509 A1
(43) Pub. Date: Feb. 29, 2024

(54) CHIRAL PHOSPHORIC ACIDS IMMOBILIZED ON SOLID SUPPORT FOR THE SELECTIVE PROTECTION OF HYDROXYL GROUPS

(71) Applicant: REGENTS OF THE UNIVERSITY OF MICHIGAN, Ann Arbor, MI (US)

(72) Inventors: Pavel Nagorny, Ann Arbor, MI (US); Iaroslav Khomutnyk, Ann Arbor, MI (US); Sibin Wang, Ann Arbor, MI (US); Oleksii Zhelavskyi, Ann Arbor, MI (US)

(21) Appl. No.: 18/232,230

(22) Filed: Aug. 9, 2023

Related U.S. Application Data
(60) Provisional application No. 63/397,220, filed on Aug. 11, 2022.

(51) Int. Cl. B01J 31/02; B01J 31/06; B01J 35/02; B01J 37/02; C07B 51/00; C07D 309/10; C07D 309/30; C07D 493/04

(52) U.S. Cl. CPC B01J 31/0258; B01J 31/06; B01J 35/026; B01J 37/0219; B01J 37/0221; C07B 51/00; C07D 309/10; C07D 309/30; C07D 493/04

(57) ABSTRACT

Provided herein are immobilized chiral phosphoric acids and methods of using immobilized chiral phosphoric acids as catalysts for protecting group reactions.

### CROSS-REFERENCE TO RELATED APPLICATION

This application claims the benefit under 35 U.S.C. § 119(e) of U.S. Provisional Patent Application No. 63/397,220, filed Aug. 11, 2022, the entire disclosure of which is incorporated herein by reference.

### STATEMENT OF GOVERNMENT SUPPORT

This invention was made with government support under grant numbers U01 GM125274 and R35 GM136341 from the National Institutes of Health. The government has certain rights in the invention.

### FIELD

The present disclosure relates to chiral phosphoric acids supported on a solid substrate and the use thereof. In particular, the disclosure relates to the use of immobilized chiral phosphoric acids as catalysts for selective protection of hydroxyl groups of cyclic polyols, including saccharides and saccharide-derived polyols.

### BACKGROUND

Carbohydrates are essential molecules that not only serve as immediate energy sources but also are associated with numerous biological activities. Accordingly, studies of carbohydrates have been the focus of many ongoing investigations in the fields of organic chemistry, biochemistry, and drug discovery. However, in many instances, gaining access to complex oligosaccharides and glycoconjugates has been a bottleneck for the exploration of their biological and medicinal properties. Complex oligosaccharides are comprised of simpler monosaccharides, and not surprisingly, modern synthetic approaches to oligosaccharides strongly rely on the ability to access differentially protected building blocks. Synthesis of such differentially protected monosaccharides often requires multiple-step sequences to differentiate numerous hydroxyl groups, and such approaches may suffer from low yields and tedious purification due to the formation of undesirable regioisomers throughout these sequences.

To address the challenges associated with the selective functionalization of monosaccharides, numerous methods, including single-pot functionalization approaches, have been previously developed. Notably, the majority of such methods rely on reagents and catalysts that discriminate between the axial and the equatorial hydroxyl groups, while only a few methods exist for the selective differentiation of equatorial hydroxyl groups that are in similar steric and electronic environments. While possible with achiral reagents or catalysts, such transformations often depend on electrophile and substrate structure and lack generality. In particular, selective protection of a single hydroxyl group, such as an acetal within a sugar-derived diol possessing hydroxyl groups in similar steric and electronic environments, represents a challenge. Acid-catalyzed acetal formation is known to proceed through the intermediacy of highly reactive oxocarbenium ions, which are known to react with nucleophiles indiscriminately. The resultant acetals are also acid labile, and equilibration and isomerization leading to the most thermodynamically stable product under the reaction conditions is not uncommon. Mixed acetal protecting groups such as 1-methoxycyclohexyl (MOC) and 2-methoxy-2-propyl (MOP) are very useful in organic synthesis and carbohydrate chemistry but are very sensitive to acids and may easily hydrolyze and/or undergo the formation of 1,3-dioxolanes and 1,3-dioxanes.

The use of asymmetric catalysts to achieve site-selective functionalization of sugar-derived polyols has received significant attention. Many efforts have focused on exploring asymmetric catalysts for selective acylation reactions, and chiral catalyst-controlled site-selective phosphorylation, thiocarbonylation, sulfonation, silylation, acetalization, and glycosylation of sugar-derived polyols have also been explored. For instance, chiral phosphoric acids have been used to achieve regio- and stereo-selective acetalization of sugar-derived polyols. In particular, 1,1′-bi-2-naphthol (BINOL)-derived chiral phosphoric acids (CPAs), including BINOL-derived CPA's bound to a solid support, have been used as catalysts to direct regioselective acetalization of carbohydrate-derived 2,3-diols.

### SUMMARY

The present disclosure relates to a SPINOL-based chiral phosphoric acid compound that is bound to a solid support and to methods of making same. The immobilized compounds of the disclosure can be represented by Formula (I)

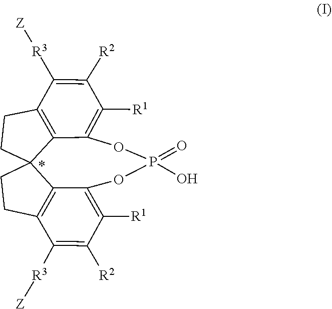

wherein each R₁ is independently selected from H; halogen; cyano; nitro; C₁₋₆ alkyl, unsubstituted or substituted with one or more substituents selected from halo, OH, substituted or unsubstituted aryl, substituted or unsubstituted heteroaryl, NR⁵R⁶, and C(═O)R⁷, wherein R⁷ is selected from OH, O(C₁₋₆ alkyl), and C₁₋₆ alkyl; C₃₋₆ cycloalkyl, unsubstituted or substituted with one or more substituents selected from halo, OH, substituted or unsubstituted aryl, and substituted or unsubstituted heteroaryl; aryl or heteroaryl, unsubstituted or substituted with one or more R⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R⁹, and NR¹⁰R¹¹; C(═O)R¹²; NR¹³R¹⁴; and OR¹⁷, wherein R¹⁷ is selected from H, C₁₋₆ alkyl, C₁₋₆ haloalkyl, C₃₋₆ cycloalkyl, substituted or unsubstituted aryl, and substituted or unsubstituted heteroaryl; and silyl, unsubstituted or substituted with one, two, or three substituents independently selected from aryl and C₁₋₆ alkyl; wherein each R⁵, R⁶, R¹⁰, R¹¹, R¹³, and R¹⁴ is independently selected from H, unsubstituted C₁₋₆ alkyl, and C₁₋₆ alkyl substituted with one or more substituents selected from OH, halo, NH₂, aryl, and heteroaryl; wherein each R⁷, R⁹, and R¹² is independently selected from OH, unsubstituted or substituted O(C₁₋₆ alkyl), unsubstituted or substituted C₁₋₆ alkyl, and unsubstituted or substituted NH₂; each R² is independently selected from H, OH, halo, NH₂, C₁₋₆ alkyl, C₁₋₆ haloalkyl, substituted or unsubstituted aryl, and substituted or unsubstituted heteroaryl; each R³ is independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing; Z denotes a solid support; and * denotes a center of chirality.

The present disclosure also relates to a method of using an immobilized chiral phosphoric acid according to Formula (I) to form a polyol having a protected hydroxyl group.

The present disclosure also relates to a method of using an immobilized chiral phosphoric acid according to Formula (I) and an immobilized BINOL-derived chiral phosphoric acid to form a polyol having multiple protected hydroxyl groups.

### DETAILED DESCRIPTION

Disclosed herein are chiral phosphoric acid compounds based on SPINOL (1,1-spirobiindane-7,7-diol) which are bound to a solid support, i.e., immobilized SPINOL-based chiral phosphoric acids. Immobilized chiral phosphoric acids according to the disclosure are useful as regioselective catalysts for hydroxyl protecting group reactions, such as acetalization, on polyols including but not limited to sugars (i.e., saccharides) and sugar-derived polyols. In particular, immobilized chiral phosphoric acids according to the disclosure were used to selectively protect equatorially-oriented hydroxyl group on cyclic polyols over adjacent equatorially-oriented hydroxyl groups. The immobilized chiral phosphoric acids can be recovered after use and reused for multiple cycles of protecting group reactions with minimal reduction in yield or selectivity.

The immobilized SPINOL-based chiral phosphoric acids (also referred to herein as “immobilized catalysts”) and methods of the disclosure can provide one or more advantages, including high yield and high regioselectivity for catalysis of protecting group reactions, straightforward recovery of the immobilized catalyst after use, and/or minimal reduction in yield or selectivity when the immobilized catalyst is reused for multiple protecting group reaction cycles. In particular, the immobilized SPINOL-based chiral phosphoric acids of the disclosure enabled regioselective protecting group reactions on various saccharide-derived 1,2-diols to proceed at high yield with regioselectivities of up to >25:1 rr (regioselectivity ratio). The immobilized catalysts were easily recycled and reused for multiple times for protecting group reactions, using catalytic loadings as low as 0.1 mol %. The performance of the immobilized catalysts was generally found to be superior to the performance of the corresponding unsupported variants.

Compounds disclosed herein may be identified either by their chemical structure and/or chemical name. When the chemical structure and chemical name conflict, the chemical structure is determinative of the identity of the compound.

### Chemical Definitions

As used herein, “halogen” and “halo” refer to F, Cl, Br, or I. As used herein, “cyano” refers to a —CN group. As used herein, “nitro” refers to a —NO₂ group.

As used herein, “hydroxy” or “hydroxyl” refers to a —OH group.

As used herein, “alkoxy” or “alkoxyl” refers to an —O-alkyl group. As used herein, “aryloxy” or “aryloxyl” refers to an —O-aryl group.

As used herein, “silyl” refers to a —SiH₃ group wherein one, two, or three hydrogens can be replaced with an alkyl, cycloalkyl, or aryl group. As used herein, “silyl ether” refers to an —O—SiH₃ group, wherein one, two, or three hydrogens can be substituted with an alkyl, cycloalkyl, or aryl group.

As used herein, “carboxy” or “carboxyl” refers to a —C(═O)OH group and “carboxylate” refers to a —C(═O)O⁻ group. A carboxylate group can be associated with an alkali metal or alkaline earth metal cation.

As used herein, “amino” refers to a —NH₂ group, wherein one or both hydrogens can be substituted with an alkyl, cycloalkyl, or aryl group.

As used herein, the term “alkyl” refers to straight chained and branched saturated hydrocarbon groups containing one to thirty carbon atoms, for example, one to twenty carbon atoms, or one to ten carbon atoms. The term Cn means the alkyl group has “n” carbon atoms. For example, C₄ alkyl refers to an alkyl group that has 4 carbon atoms. C₁₋₇ alkyl refers to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. For example, a haloalkyl group is an alkyl group that is substituted with one or more halo atoms, and can be perhalogenated (i.e., each hydrogen atom of the alkyl group is substituted with a halo atom).

As used herein, “alkylene” refers to an alkyl group wherein two hydrogens are removed to provide a divalent radical. When a range or number of carbons is provided for a particular “alkylene” group, it is understood that the range or number refers to the range or number of carbons in the linear carbon divalent chain. In embodiments, alkylene can be selected from methylene (—CH₂—), ethylene (—CH₂CH₂—), propylene (—CH₂CH₂CH₂—), and butylene (—CH₂CH₂CH₂CH₂—).

As used herein, the term “heteroalkyl” is defined similarly as alkyl, except the chain further contains one to three heteroatoms independently selected from oxygen, nitrogen, or sulfur. Nonlimiting examples of heteroalkyl groups include methoxy (—O-methyl), ethoxy (—O-ethyl), propoxy (—O-propyl), and butoxy (O-n-butyl, O-sec-butyl, or O-t-butyl).

As used herein, the term “cycloalkyl” refers to an aliphatic cyclic hydrocarbon group containing three to eight carbon atoms (e.g., 3, 4, 5, 6, 7, or 8 carbon atoms). The term Cn means the cycloalkyl group has “n” carbon atoms. For example, C₅ cycloalkyl refers to a cycloalkyl group that has 5 carbon atoms in the ring. C₅₋₈ cycloalkyl refers to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group.

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylyl, indanyl, indenyl, anthracenyl, fluorenyl, tetralinyl. Unless otherwise indicated, an aryl group can be an unsubstituted aryl group or a substituted aryl group.

As used herein, “arylene” refers to an aryl group wherein two hydrogens are removed to provide a divalent radical. For example, arylene can be phenylene or naphthylene. Unless otherwise indicated, an arylene group can be unsubstituted or substituted.

As used herein, the term “heteroaryl” refers to monocyclic or polycyclic (e.g., fused bicyclic and fused tricyclic) aromatic ring systems, wherein one to four-ring atoms are selected from oxygen, nitrogen, or sulfur, and the remaining ring atoms are carbon, said ring system being joined to the remainder of the molecule by any of the ring atoms. Nonlimiting examples of heteroaryl groups include, but are not limited to, pyridyl, pyridazinyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thienyl, quinolinyl, isoquinolinyl, benzoxazolyl, benzimidazolyl, benzofuranyl, benzothiazolyl, triazinyl, triazolyl, purinyl, pyrazinyl, purinyl, indolinyl, phthalzinyl, indazolyl, quinolinyl, isoquinolinyl, cinnolinyl, quinazolinyl, naphthyridinyl, pyridopyridinyl, indolyl, 3H-indolyl, pteridinyl, and quinooxalinyl. Unless otherwise indicated, a heteroaryl group can be an unsubstituted heteroaryl group or a substituted heteroaryl group.

As used herein, “heteroarylene” refers to a heteroaryl group wherein two hydrogens are removed to provide a divalent radical. Unless otherwise indicated, a heteroarylene group can be unsubstituted or substituted.

As used herein, the term “cyclic” refers to any ring structure comprising a cycloalkyl, heterocycloalkyl, aryl, heteroaryl, cycloalkenyl, heterocycloalkenyl, or a combination thereof. Unless otherwise indicated, a cyclic group can be an unsubstituted or a substituted cyclic group.

A “substituted” functional group (e.g., a substituted alkyl, alkylenyl, cycloalkyl, aryl, or heteroaryl) is a functional group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkenyl, alkynyl, ether, aryl, heteroaryl, heterocycloalkyl, hydroxyl, oxy (or oxo), alkoxy, ester, thioester, acyl, carboxyl, cyano, nitro, amino, sulfhydryl, and halo. When a substituted alkyl group includes more than one non-hydrogen radical, the substituents can be bound to the same carbon or two or more different carbon atoms.

As used herein, a “peroxy” compound is a compound containing an O—O bond. As used herein, an “azo” compound is a compound containing an N═N bond.

As used herein, a protecting group is a readily removable group that is not necessarily a constituent of the desired end product of a chemical reaction or series of chemical reactions. In general, a functional group on a molecule can be converted to a protecting group to enable a subsequent reaction under conditions that would cause the functional group to react or degrade. In general, protecting groups can be removed readily (i.e., without the occurrence of undesired secondary reactions), for example by solvolysis, reduction, or photolysis, to retrieve the original functional group.

As used herein, a protecting group reaction is a reaction that installs a protecting group on a substrate molecule. Generally, a protecting group reaction involves admixing at least a substrate molecule and a protecting group compound under conditions suitable to react the protecting group compound with a substituent on the substrate molecule to generate a protecting group. As used herein, a hydroxyl protecting group reaction is a protecting group reaction in which the substituent on the substrate molecule that reacts with the protecting group compound is a hydroxyl group.

### Chiral Phosphoric Acids

The present disclosure relates to a SPINOL-based chiral phosphoric acid compound that is bound to a solid support and to methods of making same. The immobilized compounds of the disclosure can be represented by Formula (I)

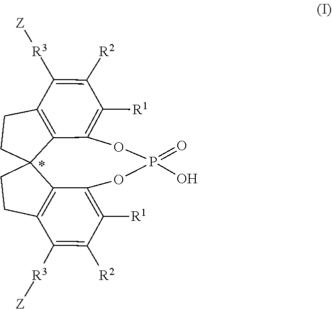

wherein each R¹ is independently selected from H; halogen; cyano; nitro; C₁₋₆ alkyl, unsubstituted or substituted with one or more substituents selected from halo, OH, substituted or unsubstituted aryl, substituted or unsubstituted heteroaryl, NR⁵R⁶, and C(═O)R⁷, wherein R⁷ is selected from OH, O(C₁₋₆ alkyl), and C₁₋₆ alkyl; C₃₋₆ cycloalkyl, unsubstituted or substituted with one or more substituents selected from halo, OH, substituted or unsubstituted aryl, and substituted or unsubstituted heteroaryl; aryl or heteroaryl, unsubstituted or substituted with one or more R⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R⁹, and NR¹⁰R¹¹; C(═O)R¹²; NR¹³R¹⁴; and OR¹⁷, wherein R¹⁷ is selected from H, C₁₋₆ alkyl, C₁₋₆ haloalkyl, C₃₋₆ cycloalkyl, substituted or unsubstituted aryl, and substituted or unsubstituted heteroaryl; and silyl, unsubstituted or substituted with one, two, or three substituents independently selected from aryl and C₁₋₆ alkyl; wherein each R⁵, R⁶, R¹⁰, R¹¹, R¹³, and R¹⁴ is independently selected from H, unsubstituted C₁₋₆ alkyl, and C₁₋₆ alkyl substituted with one or more substituents selected from OH, halo, NH₂, aryl, and heteroaryl; wherein each R⁷, R⁹, and R¹² is independently selected from OH, unsubstituted or substituted O(C₁₋₆ alkyl), unsubstituted or substituted C₁₋₆ alkyl, and unsubstituted or substituted NH₂; each R² is independently selected from H, OH, halo, NH₂, C₁₋₆ alkyl, C₁₋₆ haloalkyl, substituted or unsubstituted aryl, and substituted or unsubstituted heteroaryl; each R³ is independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing; Z denotes a solid support; and * denotes a center of chirality.

In some embodiments, R¹ can be H. In some embodiments, R¹ can be cyano. In some embodiments, R¹ can be nitro. In some embodiments, R¹ can be C₁₋₆ alkyl, unsubstituted or substituted with one or more substituents selected from halo, OH, substituted or unsubstituted aryl, substituted or unsubstituted heteroaryl, NR⁵R⁶, and C(═O)R⁷, wherein R⁵ and R⁶ are independently selected from H, unsubstituted C₁₋₆ alkyl, and C₁₋₆ alkyl substituted with one or more substituents selected from OH, halo, NH₂, aryl, and heteroaryl, and wherein R⁷ is selected from OH, unsubstituted or substituted O(C₁₋₆ alkyl), unsubstituted or substituted C₁₋₆ alkyl, and unsubstituted or substituted NH₂. In some embodiments, R¹ can be C₃₋₆ cycloalkyl, unsubstituted or substituted with one or more substituents selected from halo, OH, substituted or unsubstituted aryl, and substituted or unsubstituted heteroaryl. In some embodiments, R¹ can be aryl or heteroaryl, unsubstituted or substituted with one or more R⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R⁹, and NR¹⁰R¹¹, wherein R⁹ is selected from OH, unsubstituted or substituted O(C₁₋₆ alkyl), unsubstituted or substituted C₁₋₆ alkyl, and unsubstituted or substituted NH₂, and wherein R¹⁰ and R¹¹ are independently selected from independently selected from H, unsubstituted C₁₋₆ alkyl, and C₁₋₆ alkyl substituted with one or more substituents selected from OH, halo, NH₂, aryl, and heteroaryl. In some embodiments, R¹ can be is C(═O)R¹², wherein R¹² is independently selected from OH, unsubstituted or substituted O(C₁₋₆ alkyl), unsubstituted or substituted C₁₋₆ alkyl, and unsubstituted or substituted NH₂. In some embodiments, R¹ can be NR¹³R¹⁴, wherein R¹³ and R¹⁴ are independently selected from H, unsubstituted C₁₋₆ alkyl, and C₁₋₆ alkyl substituted with one or more substituents selected from OH, halo, NH₂, aryl, and heteroaryl. In some embodiments, R¹ can be OR¹⁷, wherein R¹⁷ is selected from H, C₁₋₆ alkyl, C₁₋₆ haloalkyl, C₃₋₆ cycloalkyl, substituted or unsubstituted aryl, and substituted or unsubstituted heteroaryl. In some embodiments, R¹ can be aryl substituted with one or more C₁₋₆ haloalkyl groups. In some embodiments, R¹ is 3,5-bis(trifluoromethyl)phenyl.

In some embodiments, R² can be H. In some embodiments, R² can be OH. In some embodiments, R² can be C₁₋₆ alkyl. In some embodiments, R² can be substituted or unsubstituted aryl.

In some embodiments, R³ can be C₁₋₆ alkylene. In some embodiments, R³ can be phenylene. In some embodiments, R³ can be alkylenephenylene. In some embodiments, R³ can be 4-(C₁₋₆ alkylene)phenylene. In some embodiments, R³ can be 4-(ethylene)phenylene.

In embodiments, R¹ is 3,5-bis(trifluoromethyl)phenyl, R² is H, R³ is phenylene, and Z is a polymer bead comprising poly(styrene-co-divinylbenzene). In embodiments, R¹ is 3,5-bis(trifluoromethyl)phenyl, R² is H, R³ is 4-(ethylene)phenylene, and Z is a polymer bead comprising poly(styrene-co-divinylbenzene).

### Preparation of Unsupported Chiral Phosphoric Acids

In general, unsupported SPINOL-based chiral phosphoric acids according to the disclosure can be prepared by admixing a SPINOL-based chiral diol (i.e., 1,1-spirobiindane-7,7-diol, optionally containing substituents at one or more of the R¹, R², and R³ positions as defined in Formula (I)), phosphoryl chloride (POCl₃), and a solvent under conditions sufficient to form a SPINOL-based chiral phosphoric acid. Synthesis of unsupported SPINOL-based chiral phosphoric acids is described in Wang et al., J. Am. Chem. Soc., 2021, 143 (44), the entire contents of which are incorporated herein by reference.

### Preparation of Immobilized Chiral Phosphoric Acids

In general, immobilized chiral phosphoric acids according to Formula (I) can be prepared by admixing an unsupported SPINOL-based chiral phosphoric acid that has been functionalized with one or more polymerizable groups in the R³ position as shown in Formula (I) and one or more polymerizable monomers under conditions sufficient to form a solid support in the form of polymer particles to which the chiral phosphoric acid is covalently bound. The polymerizable groups provided on the unsupported chiral phosphoric acid can include, but are not limited to, vinyl, allyl, and vinylphenyl groups. In embodiments, the chiral phosphoric acid is functionalized with one or more vinylphenyl groups.

In general, the immobilized chiral phosphoric acid is formed via a two-phase polymerization process, wherein an aqueous solution comprising a water-soluble stabilizing agent is admixed with a solution comprising one or more polymerizable monomers and an unsupported chiral phosphoric acid to form a two-phase mixture, a polymerization initiator is added to the two-phase mixture to form an initiated two-phase mixture, and the initiated two-phase mixture is mixed and heated under conditions sufficient to cause reaction of the polymerizable monomers and the unsupported chiral phosphoric acid and form the immobilized chiral phosphoric acid.

In embodiments, the water-soluble stabilizing agent is selected from poly(vinyl alcohol), gelatin, cellulose, and cellulose derivatives. In embodiments, the water-soluble stabilizing agent comprises poly(vinyl alcohol).

In embodiments, the polymerizable monomers comprise styrene and divinylbenzene.

In embodiments, the polymerization initiator can be a peroxy or azo compound. In embodiments, the polymerization initiator can be a peroxide. In embodiments, the polymerization initiator is benzoyl peroxide.

In embodiments, the two-phase mixture is heated at a temperature between 70° C. and 110° C., or between 80° C. and 100° C., or between 90° C. and 95° C. In embodiments, the two-phase mixture is heated at 95° C.

In embodiments, the two-phase mixture is heated for up to 48 hours, or up to 42 hours, or up to 36 hours.

Alternatively, immobilized chiral phosphoric acids according to Formula (I) can be prepared by reacting a solid support with a chiral phosphoric acid that has been functionalized with one or more polymerizable groups in the R³ position as shown in Formula (I).

In embodiments, SPINOL-based chiral phosphoric acids contain substituents at the R¹ position denoted in Formula (I). Without intending to be bound by theory, it is believed that bulky substituents allow the chiral phosphoric acid to maintain its chirality and not racemize. In embodiments, R¹ can be an unsubstituted or substituted aryl group or an unsubstituted or substituted heteroaryl group. In embodiments, each R¹ is 3,5-bis(trifluoromethyl)phenyl.

### Solid Supports

In general, the solid support to which the chiral phosphoric acid compound according to the disclosure is bound can be any solid support that can immobilize the chiral phosphoric acid in a single configuration. The solid support to which the chiral phosphoric acid compound according to the disclosure is bound can be selected from, but is not limited to, polymers, clays, zeolites, and silicon-based resins. In some embodiments, the solid support comprises a polymer, a clay, a zeolite, a silicon-based resin, or a combination thereof. In some embodiments, the solid support comprises a polymer, a silicon-based resin, or a combination thereof. In some embodiments, the solid support comprises a polymer.

The solid support can be in the form of particles which are substantially spherical, substantially nonspherical, or a mix of substantially spherical and substantially nonspherical particles. As used herein, and unless specified otherwise, a particle is considered “substantially spherical” if its aspect ratio is less than or equal to 1.5, wherein aspect ratio as used herein refers to ratio of the longest dimension of the particle to the shortest dimension of the particle. Similarly, a particle is considered “substantially nonspherical” if its aspect ratio is greater than 1.5. The particles can have an average particle size in a range from 0.001 mm to 5.0 mm, or from 0.005 mm to 4.0 mm, or from 0.010 mm to 3.0 mm, or from 0.050 mm to 2.0 mm, or from 0.100 mm to 1.5 mm, or from 0.200 mm to 1.2 mm, or from 0.500 mm to 1.0 mm.

In embodiments where the solid support comprises a polymer, the solid support is hereafter also referred to as a polymer bead. Suitable polymers for the polymer bead of the disclosure include polymers resulting from polymerization of ethylenically unsaturated monomers and derivatives of such polymers, including but not limited to polystyrene, poly(styrene sulfonate), substituted polystyrenes, polyacrylic acid, polyacrylates, polymethacrylic acid, polymethacrylates, poly(4-vinylpyridine), polyethylene, polypropylene, polyolefins, polyvinyl acetate, polyvinyl alcohol, and copolymers of the foregoing. In some embodiments, the polymer bead comprises polystyrene, substituted polystyrene, polyacrylates, or polymethacrylates. In some embodiments, the polymer bead comprises polystyrene.

In embodiments where the solid support comprises a polymer, the solid support can be in the form of cross-linked polymer beads. Cross-linked polymer beads can be prepared by including a polymerizable cross-linking agent in the preparation of the polymer bead. Cross-linking agents are well known in the art and are generally considered molecules containing two or more reactive groups, such as unsaturated groups, that can couple or tether two polymer chains. Suitable cross-linking agents include but are not limited to divinylbenzene, allyl methacrylate, ethylene glycol dimethacrylate, and trimethylolpropane trimethacrylate. In embodiments, the cross-linking agent is divinylbenzene.

In embodiments, the solid support comprises a cross-linked polymer bead comprising poly(styrene-co-divinylbenzene).

Polymer beads according to the disclosure can be prepared by any suitable polymerization method, including but not limited to suspension polymerization, anionic polymerization, cationic polymerization, and emulsion polymerization. In embodiments, the polymer bead is prepared by suspension polymerization.

In general, the chiral phosphoric acid can be coupled to the solid support. As used herein, “couple,” “coupling,” and other variations of “couple” encompasses any one or more of covalent bond formation, hydrogen bond formation, ionic bond formation (e.g., electrostatic attraction), and van der Waals interactions, for example, through which the chiral phosphoric acid can adsorb to/adhere to/couple to/associate with the solid support. In embodiments in which the chiral phosphoric acid compound is covalently bound to a solid support, the group coupling the chiral phosphoric acid compound to the solid support can be a group on the solid support independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing.

### Substrates for Protecting Group Reactions

Chiral phosphoric acids according to the disclosure are useful as catalysts for selectively protecting a hydroxyl group on substrate molecules comprising one or more hydroxyl groups. In embodiments, substrates for protecting group reactions can include but are not limited to monohydric alcohols, diols, and higher polyols, including but not limited to carbohydrates, cyclic polyols, and natural or synthetic saccharides and derivatives thereof. In embodiments, the substrate for the protecting group reaction is a cyclic polyol.

As used herein, “saccharides” include monosaccharides, disaccharides, oligosaccharides, polysaccharides, and derivatives thereof. In embodiments, the substrate for the protecting group reaction is a monosaccharide or a derivative thereof. In embodiments, the substrate for the protecting group reaction is a disaccharide or a derivative thereof. In embodiments, the substrate for the protecting group reaction is an oligosaccharide or a derivative thereof. In embodiments, the substrate for the protecting group reaction is a polysaccharide or a derivative thereof. In embodiments, the substrate for the protecting group reaction is glucose, fructose, galactose, mannose, fucose, ribose, xylose, rhamnal, or a derivative thereof.

Saccharides can exist in open-chain (i.e., acyclic) or cyclic form. The cyclic form of a saccharide can contain one or more pyranose (6-membered) or furanose (5-membered) rings. The numbering convention for the cyclic form of a saccharide or cyclic polyol comprising a 6-membered ring adopting a chair configuration is shown in Scheme 1. Regioselectivity ratios (rr) for protecting group reactions on cyclic saccharides as described herein are expressed using this numbering convention. For instance, a C2:C3 regioselectivity ratio for a protecting group reaction on a cyclic saccharide is a ratio of the amount of reaction product in which the hydroxyl group in the C2 position is protected to the amount of reaction product in which the hydroxyl group in the C3 position is protected.

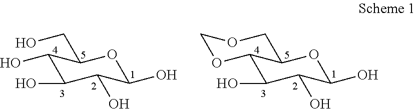 [SMILES: OCC1OC(O)C(O)C(O)C1O ; OC1OC2COCOC2C(O)C1O]

As used herein, derivatives of saccharides include molecules that comprise a saccharide structure and further comprise one or more substituents on the saccharide structure. Derivatives of saccharides include molecules comprising one or more rings fused to a ring of a cyclic saccharide, such as the fused-ring structure shown in Scheme 1, right, and substituted derivatives thereof.

In embodiments, the substrate for the protecting group reaction is a cyclic polyol having a 2-position and a 3-position. In embodiments, the cyclic polyol having a 2-position and a 3-position has hydroxyl groups in the 2- and 3-positions. In embodiments, the hydroxyl groups in the 2- and 3-positions of the cyclic polyol are in an equatorial conformation.

### Protecting Group Reactions

Immobilized chiral phosphoric acids according to the disclosure are useful as catalysts for hydroxyl protecting group reactions. Hydroxyl protecting group reactions can include but are not limited to reactions of hydroxyl groups to form an ether, ester or acetal protecting group. Acetal protecting groups can include but are not limited to 2-methoxy-2-propyl and 1-methoxycyclohexyl. Ether protecting groups can include but are not limited to methoxymethyl (MOM), benzyl, p-methoxybenzyl, trityl, t-butyl, tetrahydropyranyl, and silyl. Ester protecting groups can include but are not limited to acetyl, trimethylacetyl (pivaloyl), trifluoroacetyl, and benzoyl. In embodiments, immobilized compounds according to the disclosure can be used as catalysts for protecting group reactions that form acetal protecting groups. In embodiments, immobilized compounds according to the disclosure can be used as catalysts for protecting group reactions that form 2-methoxy-2-propyl protecting groups. In embodiments, immobilized compounds according to the disclosure can be used as catalysts for protecting group reactions that form 1-methoxycyclohexyl protecting groups.

In embodiments, the protecting group compound for forming an acetal protecting group is an enol ether. In embodiments, the protecting group compound is 2-methoxypropene (2-MOP). In embodiments, the protecting group compound is 1-methoxycyclohexene (1-MOC). In embodiments, the protecting group compound is 3,4-dihydropyran. In embodiments, the protecting group compound is 4-methoxy-3,6-dihydro-2H-pyran.

In general, protecting group reactions can be carried out by admixing a diol or polyol, a protecting group compound, an unsupported or immobilized chiral phosphoric acid according to the disclosure, and a solvent under conditions suitable to form a product comprising a diol or polyol having a protected hydroxyl group. The protecting group reactions can be carried out via a batch process or via a continuous flow process.

In general, the solvent for the protecting group reaction is a solvent for the polyol, the protecting group compound, or both. In embodiments, the solvent for the protecting group reaction is a solvent for both the polyol and the protecting group compound. In embodiments, the solvent comprises dichloromethane, chloroform, toluene, hexanes, diethyl ether, tetrahydrofuran, xylenes, pentanes, 1,1-dichloroethane, 1,2-dichloroethane, or a combination thereof. In embodiments, the solvent comprises dichloromethane.

In embodiments, the protecting group reaction is carried out at a temperature of about 0° C. or below. In embodiments, the protecting group reaction is carried out at a temperature of about −50° C. or below. In embodiments, the protecting group reaction is carried out at a temperature in a range of about −100° C. to 0° C., or about −78° C. to about −50° C. In some embodiments, the protecting group reaction is carried about at a temperature of about −78° C. In some embodiments, the protecting group reaction is carried out at a temperature of about −50° C. In some embodiments, the protecting group compound is 2-MOP and the protecting group reaction is carried out at a temperature of about −78° C. In some embodiments, the protecting group compound is 1-MOP and the protecting group reaction is carried out at a temperature of about −50° C. Without intending to be bound by theory, it is believed that carrying out a protecting group reaction at a temperature in a range of about −100° C. to 0° C. provides improved stereoselectivity compared to carrying out the same reaction at a temperature greater than about 0° C.

In embodiments, protecting group reactions are carried out by mixing for up to 36 hours, or up to 24 hours, or up to 18 hours.

In embodiments, protecting group reactions can be carried out wherein the center of chirality of the SPINOL-based chiral phosphoric acid according to the disclosure is in an (S) conformation. In embodiments, protecting group reactions can be carried out wherein the center of chirality of the SPINOL-based chiral phosphoric acid according to the disclosure is in an (R) conformation.

In general, the unsupported catalyst or immobilized catalyst is provided to a protecting group reaction in catalytic amounts. In various embodiments, the amount of unsupported catalyst or immobilized catalyst is in a range from 0.01 mol % to 5 mol %, or 0.1 mol % to 2 mol %, or 0.2 mol % to 1.5 mol %, or 0.5 mol % to 1.0 mol %, based on the amount of polyol in the protecting group reaction. In embodiments, the amount of catalyst provided to the protecting group reaction is 2 mol %. In embodiments, the amount of catalyst provided to the protecting group reaction is 0.5 mol %. In embodiments, the amount of catalyst provided to the protecting group reaction is 0.1 mol %.

In general, protecting group reactions can be carried out in an oxygen-free atmosphere. In some embodiments, protecting group reactions can be carried out in a nitrogen environment, and argon environment, or a combination thereof. In the specific examples disclosed herein, protecting group reactions on various diols and polyols were carried out in a nitrogen environment.

In embodiments, the method according to the disclosure provides a product comprising a cyclic polyol having a protected hydroxyl group. In embodiments, the cyclic polyol having a protected hydroxyl group has a 2-position and a 3-position. In embodiments, the product comprises an amount of cyclic polyol having a protected hydroxyl group in the 3-position that is at least 20 times greater than the amount of cyclic polyol having a protected hydroxyl group in the 2-position comprising the product.

Regioselectivity ratios (rr) for protecting group reactions were determined by ¹H NMR analysis, as described in Wang et al., J. Am. Chem. Soc., 2021, 143 (44).

### Methods of Protecting Multiple Hydroxyl Groups

Further contemplated is the use of multiple immobilized chiral phosphoric acids to protect multiple hydroxyl groups of a polyol.

Chiral phosphoric acids based on BINOL (1,1′-bi-2-naphthol), including unsupported BINOL-based chiral phosphoric acids and BINOL-based chiral phosphoric acids bound to a solid support, can be used as catalysts to direct regioselective protection of carbohydrate-derived 2,3-diols.

Disclosed herein are methods of protecting multiple hydroxyl groups of a polyol, the method comprising admixing a polyol comprising two or more hydroxyl groups, two or more protecting group compounds, an immobilized SPINOL-based chiral phosphoric acid according to the disclosure, an immobilized BINOL-based chiral phosphoric acid according to Formula (II),

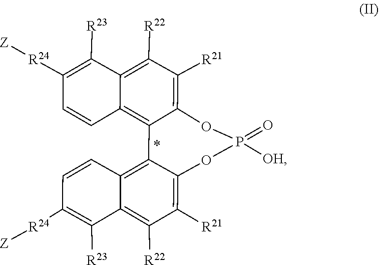

and a solvent to form a product comprising a polyol having multiple protected hydroxyl groups.

In Formula (II), each R₂₁ is independently selected from H; cyano; nitro; C₁₋₆ alkyl, unsubstituted or substituted with one or more substituents selected from halo, OH, substituted or unsubstituted aryl, substituted or unsubstituted heteroaryl, NR²⁵R²⁶, and C(═O)R²⁷; C₃₋₆ cycloalkyl, unsubstituted or substituted with one or more substituents selected from halo, OH, substituted or unsubstituted aryl, and substituted or unsubstituted heteroaryl; aryl or heteroaryl, unsubstituted or substituted with one or more R²⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R²⁹, and NR³⁰R³¹; C(═O)R³²; NR³³R³⁴; and OR³⁵, wherein R³⁵ is selected from H, substituted or unsubstituted C₁₋₆ alkyl, substituted or unsubstituted C₃₋₆ cycloalkyl, substituted or unsubstituted aryl, and substituted or unsubstituted heteroaryl; wherein each R²⁵, R²⁶, R³⁰, R³¹, R³³, and R³⁴ is independently selected from H, unsubstituted C₁₋₆ alkyl, and C₁₋₆ alkyl substituted with one or more substituents selected from OH, halo, NH₂, aryl, and heteroaryl, and wherein each R²⁷, R²⁹, and R³² is independently selected from OH, unsubstituted or substituted O(C₁₋₆ alkyl), unsubstituted or substituted C₁₋₆ alkyl, and unsubstituted or substituted NH₂.

In Formula (II), each R²² and R²³ is independently selected from H, OH, halo, NH₂, C₁₋₆ alkyl, C₁₋₆ haloalkyl, substituted or unsubstituted aryl, and substituted or unsubstituted heteroaryl.

In Formula (II), each R²⁴ is independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing.

In Formula (II), Z denotes a solid support and * denotes an axis of chirality.

In embodiments, the two or more protecting group compounds are different types of protecting group compounds. In embodiments, the two or more protecting group compounds are the same of protecting group compound.

In embodiments, the method comprises admixing the polyol comprising two or more hydroxyl groups, a protecting group compound, the compound according to Formula (II), and the solvent to form a product comprising a polyol having a protected hydroxyl group. In a related embodiment, the method further comprises mixing the product comprising a polyol having a protected hydroxyl group, a protecting group compound, and the immobilized SPINOL-based chiral phosphoric acid according to the disclosure to form a product comprising a polyol having multiple protected hydroxyl groups.

In embodiments, the method comprises admixing the polyol comprising two or more hydroxyl groups, a protecting group compound, the immobilized SPINOL-based chiral phosphoric acid according to the disclosure, and the solvent to form a product comprising a polyol having a protected hydroxyl group. In a related embodiment, the method further comprises mixing the product comprising a polyol having a protected hydroxyl group, a protecting group compound, and a compound according to Formula (II) to form a product comprising a polyol having multiple protected hydroxyl groups.

In embodiments, the method comprises simultaneously admixing the immobilized SPINOL-based chiral phosphoric acid according to the disclosure and the compound according to Formula (II) with the polyol comprising two or more hydroxyl groups, the two or more protecting group compounds, and the solvent.

In general, the solvent is a solvent for the polyol, the two or more protecting group compounds, or both. In embodiments, the solvent is a solvent for both the polyol and the two or more protecting group compounds. In embodiments, the solvent comprises dichloromethane, chloroform, toluene, hexanes, diethyl ether, tetrahydrofuran, xylenes, pentanes, 1,1-dichloroethane, 1,2-dichloroethane, or a combination thereof. In embodiments, the solvent comprises dichloromethane.

In embodiments, the method is carried out at a temperature of about 0° C. or below. In embodiments, the method is carried out at a temperature of about −50° C. or below. In embodiments, the method is carried out at a temperature in a range of about −100° C. to 0° C., or about −78° C. to about −50° C. In some embodiments, the method is carried about at a temperature of about −78° C. In some embodiments, the method is carried out at a temperature of about −50° C. Without intending to be bound by theory, it is believed that carrying out the method at a temperature in a range of about −100° C. to 0° C. provides improved stereoselectivity compared to carrying out the method at a temperature greater than about 0° C.

In embodiments, the center of chirality of the SPINOL-based chiral phosphoric acid according to the disclosure is in an (S) conformation and the axis of chirality of the BINOL-based chiral phosphoric acid in an (R) conformation.

In embodiments, the SPINOL-based chiral phosphoric acid according to the disclosure and the BINOL-based chiral phosphoric acid according to Formula (II) are each provided in an amount in a range of 0.1 mol % to 2 mol % based on the amount of polyol comprising two or more hydroxyl groups.

In embodiments, the two or more protecting group compounds are enol ethers. In embodiments, the two or more protecting group compounds are independently selected from 3,4-dihydropyran, 2-methoxypropene, 1-methoxycyclohexene, and 4-methoxy-3,6-dihydro-2H-pyran.

In embodiments, the polyol comprising two or more hydroxyl groups is a cyclic polyol. In embodiments, the cyclic polyol comprises a monosaccharide or an oligosaccharide. In embodiments, the cyclic polyol comprises a monosaccharide. In embodiments, the monosaccharide is selected from glucose, galactose, mannose, fucose, and rhamnal, and derivatives thereof. In embodiments, the monosaccharide is selected from D-glucose, D-galactose, D-mannose, L-fucose, and L-rhamnal, and derivatives thereof.

Protecting group reactions for protecting one or multiple hydroxyl groups as described herein can be carried out via a continuous flow process, such as a process as described below. One or more immobilized catalysts according to the disclosure can be loaded into a column. Optionally, prior to carrying out the protecting group reaction, a suitable solvent can be passed through the column to swell the solid support comprising the immobilized catalyst(s). One or more solutions comprising reactants for the protecting group reaction (i.e., a protecting group compound and a diol or polyol) and a solvent can then be passed through the column, such that the reactants and catalyst are admixed within the column, under conditions suitable to form a product comprising a diol or polyol having a protected hydroxyl group. The flow rate of reactants through the column can be adjusted, for instance to affect properties of the reaction products (e.g., yield, regioselectivity). The crude product mixture can be collected from the output from the column, and reaction products can be isolated from the crude product mixture and purified according to known methods.

### EXAMPLES

BINOL-based chiral phosphoric acid 1 was prepared according to the procedure described in Jiao et al., Angew. Chem. Int. Ed., 2008, 47 (13), 2411-2413, the contents of which are incorporated herein by reference. Immobilized BINOL-based chiral phosphoric acid 2 were prepared according to the procedure described in Wang et al., J. Am. Chem. Soc., 2021, 143 (44), 18592-18604.

### Example 1a: Synthesis of Immobilized Catalyst (R)-Ad-TRIP-PS ((R)-2)

The synthesis of immobilized BINOL-based catalyst (R)-Ad-TRIP-PS, also denoted herein as (R)-2, is described below. Structures of unsupported catalyst 1, immobilized catalyst (R)-2 and reagents and intermediates in the synthesis of (R)-2 are shown below. “1-Ad” denotes a 1-adamantyl substituent. The vertical line in the structure of (R)-2 denotes the solid support to which the chiral phosphoric acid is bound.

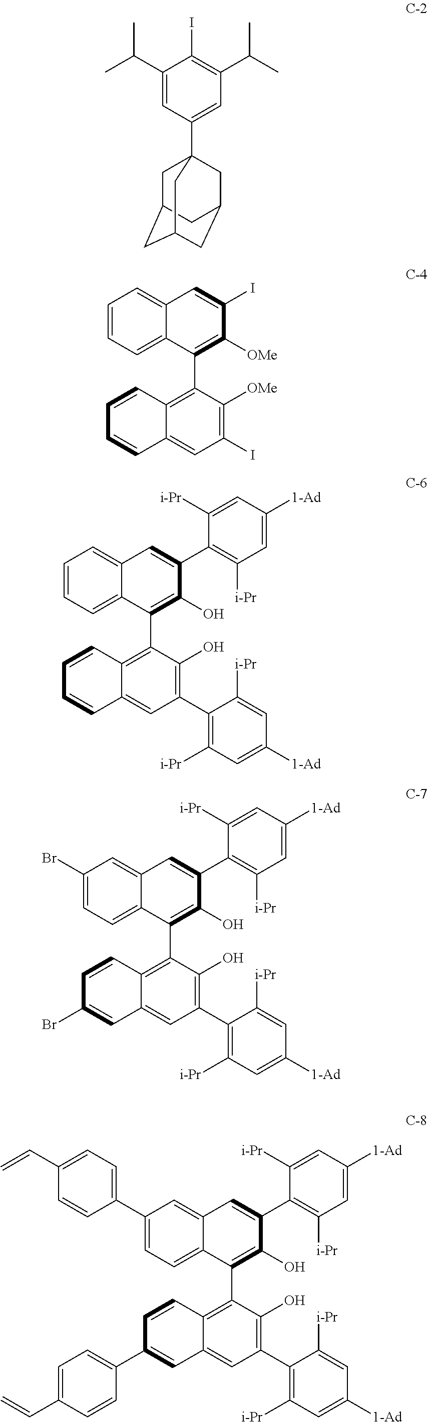

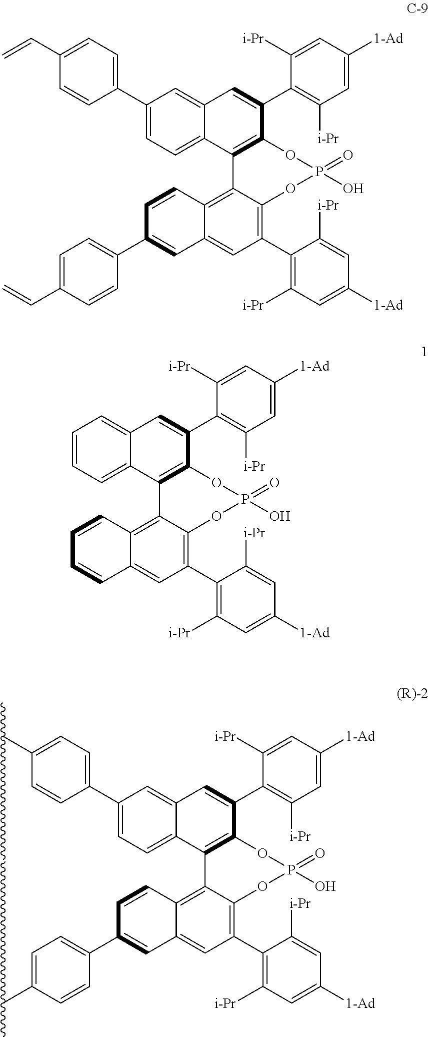

### Synthesis of C-6

Reagents C-2 and C-4 were prepared using previously reported methods, as described in Jiao et al, Angew. Chem. Int. Ed., 2008, 47 (13), 2411.

C-2 (1.43 g, 3.4 mmol) was dissolved in a solution of 30 mL pentane and 15 mL diethyl ether. The resulting solution was cooled to −78° C., and a 1.6 M solution of n-BuLi (2.1 mL, 3.4 mmol, 1.0 equiv.) was added slowly. The mixture was warmed to 0° C. with stirring over 30 minutes. The resulting mixture was cooled back to −78° C., followed by dropwise addition of a solution of ZnBr₂ (0.92 g, 4.10 mmol, 1.2 equiv.) in THF. The reaction mixture was then warmed to about 20-25° C. (room temperature) and stirred for 90 minutes. The resulting mixture was dried under reduced pressure to yield an off-white solid which was used directly without purification. The off-white solid was mixed with reagent C-4 (0.57 g, 1.00 mmol, 0.33 equiv.) and Pd(P(t-Bu)₃)₂ (3.3 mg, 68 μmol, 0.02 equiv.); the resulting mixture was dissolved in THF (5 mL) and refluxed at 80° C. for 12 hours. When the reaction was complete as determined by TLC, the reaction mixture was diluted with hexane and diethyl ether (1:1 ratio, 200 mL), then washed with 1N HCl (40 mL), washed with saturated NaHCO₃ solution (30 mL), and dried with NaCl brine and Na₂SO₄ under reduced pressure. The resulting foaming brown solid was used without further purification. The crude product was then mixed with dichloromethane (0.16 M, 6 mL) and the resulting mixture was cooled to 0° C. BBr₃ (6 equiv. 6 mL) was added dropwise, after which the mixture was warmed to room temperature and stirred for 12 hours until TLC indicated the reaction was complete. The mixture cooled to 0° C., and water (20 mL) was added slowly. The resulting organic layer was separated, and the remaining aqueous layer was extracted with dichloromethane (3×40 mL) and dried with brine and Na₂SO₄. The crude product was then purified with 5% ethyl acetate in hexane to obtain C-6 as a pale yellow product (0.59 g, 67% yield).

¹H NMR (700 MHz, chloroform-d): δ=7.86 (d, J=8.0 Hz, 2H), 7.77 (s, 2H), 7.37 (ddd, J=8.1, 5.9, 2.1 Hz, 2H), 7.30 (dd, J=6.1, 1.5 Hz, 4H), 4.91 (s, 2H), 2.86 (p, J=6.7 Hz, 2H), 2.71 (p, J=6.8 Hz, 2H), 2.12 (s, 6H), 1.99 (d, J=2.9 Hz, 12H), 1.80 (s, 12H), 1.20 (d, J=6.8 Hz, 6H), 1.10 (dd, J=8.7, 6.9 Hz, 12H), 1.03 (d, J=6.9 Hz, 6H). ¹³C NMR (176 MHz, chloroform-d): δ=151.5, 150.6, 147.3, 147.3, 133.4, 130.5, 130.0, 129.1, 129.0, 128.2, 126.5, 124.5, 123.7, 119.6, 113.2, 43.2, 36.9, 36.5, 31.0, 31.0, 29.0, 24.4, 24.4, 24.0, 23.8. IR (thin film, cm⁻¹): 3520, 2958, 2901, 2847, 1601, 1497, 1422, 1382, 1361, 1344, 1315, 1255, 1234, 1147, 1121, 746. HRMS (ESI+) (m/z): [M+NH₄]⁺ calculated for C₆₄H₇₈NO₂: 892.6027, found: 892.6011. [α]_{D}²⁵=35.1° (c=1.77, CH₂Cl₂).

### Synthesis of C-7

A solution of diol C-6 (2.1 g, 2.3 mmol) in dry DCM (12 mL) was cooled to −78° C. and a solution of Br₂ (0.25 mL, 5.3 mmol, 2.2 equiv.) in 12 mL of dry DCM was added dropwise. The bromine solution became discolored almost instantly upon addition. The reaction mixture was stirred for 2.5 hours at −78° C., then heated to room temperature and stirred for an additional 12 hours. After completion of the reaction as indicated by TLC, the reaction mixture was washed with a 10% w/w aqueous solution of NaS₂O₃ (40 mL) and extracted with DCM (3×40 mL). The organic layer was separated and washed with brine, and DCM was removed by rotary evaporation. The crude product C-7 (2.2 g, 92% yield) was used directly without purification.

¹H NMR (401 MHz, chloroform-d): δ=8.00 (d, J=2.0 Hz, 2H), 7.66 (s, 2H), 7.38 (d, J=2.0 Hz, 1H), 7.36 (d, J=2.1 Hz, 1H), 7.29 (d, J=1.9 Hz, 2H), 7.13 (s, 1H), 7.11 (s, 1H), 5.30 (s, 4H), 4.89 (s, 2H), 2.77 (q, J=6.9 Hz, 2H), 2.71-2.58 (m, 2H), 2.13 (s, 6H), 1.98 (d, J=3.0 Hz, 12H), 1.80 (s, 12H), 1.20 (d, J=6.9 Hz, 6H), 1.09 (dd, J=9.5, 6.8 Hz, 12H), 1.02 (d, J=6.8 Hz, 6H). ¹³C NMR (176 MHz, chloroform-d): δ=152.1, 150.6, 150.2, 147.5, 147.4, 146.9, 132.0, 130.3, 130.1, 130.1, 130.0, 129.3, 128.9, 126.3, 123.6, 120.8, 119.9, 117.5, 113.6, 49.3, 43.2, 43.2, 36.9, 36.8, 36.6, 36.3, 35.5, 33.7, 32.6, 31.1, 31.0, 29.7, 29.0, 29.0, 29.0, 24.4, 24.3, 24.0, 23.8, 23.2. IR (thin film, cm⁻¹): 3510, 2959, 2900, 2847, 1593, 1485, 1435, 1382, 1361, 1344, 1315, 1258, 1183, 1124, 1103, 1069, 935, 899, 873, 812. HRMS (ESI+) (m/z): [M+NH₄]⁺ calculated for C₆₄H₇₆Br₂NO₂: 1050.4222, found 1050.4224. [α]_{D}²⁵=37.2° (c=1.13, CH₂Cl₂).

### Synthesis of C-8

A solution of C-7 (310 mg, 0.3 mmol), 4-vinylphenylboronic acid (106 mg, 0.72 mmol, 2.4 equiv.), and K₂CO₃ (170 mg, 1.2 mmol, 4.0 equiv.) in 1:1 ethanol:THF 1:1 (3 mL) was degassed, followed by addition of Pd(PPh₃)₄ (52 mg, 0.045 mmol, 15 mol %). The reaction mixture was refluxed at 85° C. with stirring for 13 hours, then cooled to room temperature. Volatiles were removed using rotary evaporation, and the resulting solid residue was dissolved in dichloromethane (15 mL) and water (15 mL). The organic layer was separated, and the aqueous layer was extracted with dichloromethane (3×30 mL). The combined organic layers were then dried with brine and sodium sulfate. The organic solution was concentrated by rotary evaporation, and the resulting residue was directly purified by flash column chromatography on silica using 5% ethyl acetate in hexanes to provide C-8 (211 mg, 0.195 mmol) as a brown-yellow solid in 65% yield.

¹H NMR (500 MHz, chloroform-d): δ=8.06 (d, J=1.9 Hz, 2H), 7.83 (s, 2H), 7.71-7.63 (m, 4H), 7.62-7.56 (m, 2H), 7.53-7.47 (m, 4H), 7.40 (d, J=8.8 Hz, 2H), 7.26 (s, 4H), 6.76 (dd, J=17.6, 10.9 Hz, 2H), 5.80 (dd, J=17.6, 0.9 Hz, 2H), 5.27 (dt, J=10.9, 1.6 Hz, 2H), 4.97 (s, 2H), 2.91 (p, J=6.9 Hz, 2H), 2.75 (p, J=6.8 Hz, 2H), 2.13 (s, 6H), 1.99 (d, J=2.9 Hz, 12H), 1.88-1.72 (m, 12H), 1.25-1.20 (m, 6H), 1.16-1.09 (m, 12H), 1.05 (d, J=6.9 Hz, 6H). ¹³C NMR (176 MHz, chloroform-d): δ=151.7, 150.7, 147.4, 147.4, 140.5, 136.4, 136.1, 132.7, 130.7, 129.7, 129.6, 129.2, 127.3, 126.7, 126.1, 126.0, 125.2, 120.0, 113.8, 113.3, 43.2, 36.9, 36.5, 31.1, 31.0, 29.0, 24.4, 24.4, 24.1, 23.8. IR (thin film, cm⁻¹): 3518, 2958, 2901, 2847, 1602, 1495, 1464, 1382, 1361, 1315, 1255, 1233, 1103, 939, 904, 841. HRMS (ESI+) (m/z): [M+H]⁺ calculated for C₈₀H₈₅O₂⁺, 1077.6555, found 1077.6580. [α]_{D}²⁵=49.1° (c=1.18, CH₂Cl₂).

### Synthesis of C-9

To a stirred solution of C-8 (216 mg, 0.20 mmol) and pyridine (0.1 M, 2.0 mL) was added POCl₃ (38 μL, 0.40 mmol, 2.0 equiv.). The reaction mixture was stirred at 70° C. for 12 hours. The reaction mixture was then added to water (2 mL) and stirred continuously at 100° C. for 12 hours. After cooling to room temperature, the reaction mixture was diluted with DCM (40 mL). The organic layer was separated and the aqueous layer was extracted with DCM (3×40 mL). The organic layer was then washed with 6 N HCl (3×40 mL). The combined organic layers were then dried with brine and anhydrous sodium sulfate and concentrated under reduced pressure to obtain a pale yellow solid. This crude product was purified by column chromatography on silica gel using 2% MeOH in DCM. The obtained product was then dissolved in DCM, washed again with 6 N HCl, dried with anhydrous sodium sulfate, and concentrated under reduced pressure to obtain the free acid form of C-9 (128 mg, 57% yield).

¹H NMR (700 MHz, chloroform-d): δ=8.11 (d, J=1.9 Hz, 2H), 7.97 (s, 2H), 7.72 (d, J=8.0 Hz, 4H), 7.65 (dd, J=8.9, 1.9 Hz, 2H), 7.54 (dd, J=16.6, 8.5 Hz, 6H), 7.23-7.10 (m, 4H), 6.80 (dd, J=17.5, 10.9 Hz, 2H), 5.83 (d, J=17.6 Hz, 2H), 5.31 (d, J=10.8 Hz, 2H), 2.69 (dt, J=14.7, 7.1 Hz, 4H), 2.12 (s, 6H), 2.01-1.92 (m, 12H), 1.84-1.72 (m, 12H), 1.34-1.23 (m, 6H), 1.19-1.06 (m, 12H), 0.99 (t, J=12.0 Hz, 6H). ¹³C NMR (176 MHz, chloroform-d): δ=150.8, 147.4, 146.8, 139.9, 138.1, 137.0, 136.3, 132.9, 132.8, 131.4, 130.8, 128.0, 127.5, 126.8, 125.9, 125.7, 121.8, 119.5, 118.6, 114.2, 43.2, 43.1, 36.9, 36.4, 31.2, 31.0, 29.7, 29.1, 26.8, 25.2, 23.5, 22.8, 20.8. ³¹P NMR (283 MHz, chloroform-d): δ=2.73. IR (thin film, cm⁻¹): 2958, 2901, 2847, 1734, 1603, 1576, 1457, 1407, 1361, 1257, 1095, 1015, 974, 935, 873, 843, 732. HRMS (ESI+) (m/z): [M−H]⁻ calculated for C₈₀H₈₅O₄P⁻=1139.6113, found 1139.6100. [α]_{D}²⁵=−29.74° (c=1.06, CH₂Cl₂).

### Preparation of Immobilized Catalyst (R)-2

Solution A (aqueous phase) was prepared by charging polyvinyl alcohol (PVA, MW 31,000-35,000; 3.0 mg) to water (3.0 mL), heating to 80° C., and mixing for 60 minutes until the PVA was completely dissolved. The PVA solution was cooled to room temperature and degassed by sparging with nitrogen for 15 minutes.

Solution B (monomer phase) was prepared by mixing styrene (796 mg), divinylbenzene (80%; 28 mg), C-9 (312 mg), and toluene (2.0 mL). Styrene and divinylbenzene were washed with 3N aqueous NaOH solution, passed through an alumina plug, and dried over sodium sulfate immediately prior to use.

Solution A and Solution B were mixed in a screw thread tube, and dibenzoyl peroxide (75%, water stabilized) (13.2 mg) was added. The mixture was degassed by sparging with nitrogen for 5 minutes at room temperature and heated to 95° C. while mixing at 1100 rpm. After 36 hours, the reaction mixture was cooled to room temperature, and solid material was collected by vacuum filtration. The solid material was successively washed with water, methanol, THF, DCM, and hexane and dried under reduced pressure overnight to obtain yellow powder particles of immobilized catalyst (R)-Ad-TRIP-PS ((R)-2; 1.07 g).

Elemental analysis of (R)-2: C: calculated 89.9%, observed 88.4%; H: calculated 7.7%, observed 7.6%.

### Example 1b: Synthesis of Immobilized Catalyst (S)-SPINOL-PS ((S)-3)

Immobilized SPINOL-based catalyst (S)-SPINOL-PS, also denoted herein as (S)-3, is described below. Structures of unsupported catalyst 16, immobilized catalyst (S)-3, achiral phosphoric acid BPPA, and reagents and intermediates in the synthesis of (S)-3 are shown below. The vertical line in the structure of (S)-3 denotes the solid support to which the chiral phosphoric acid is bound.

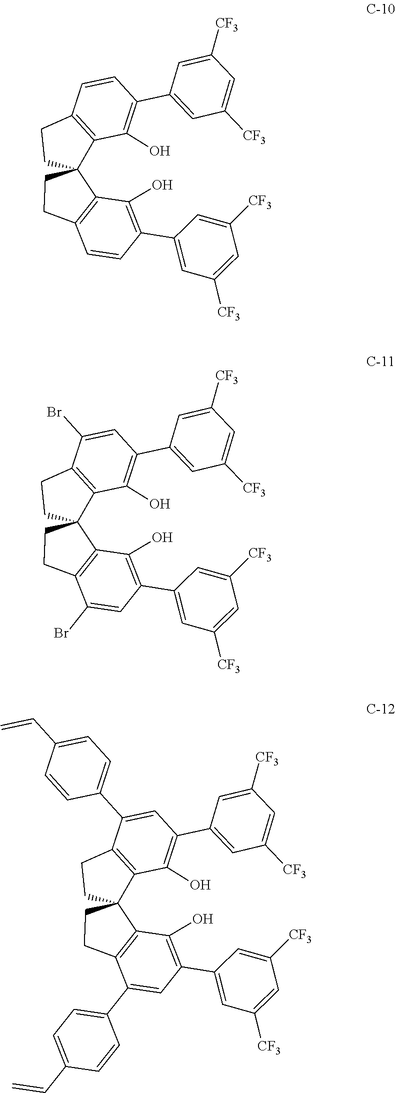

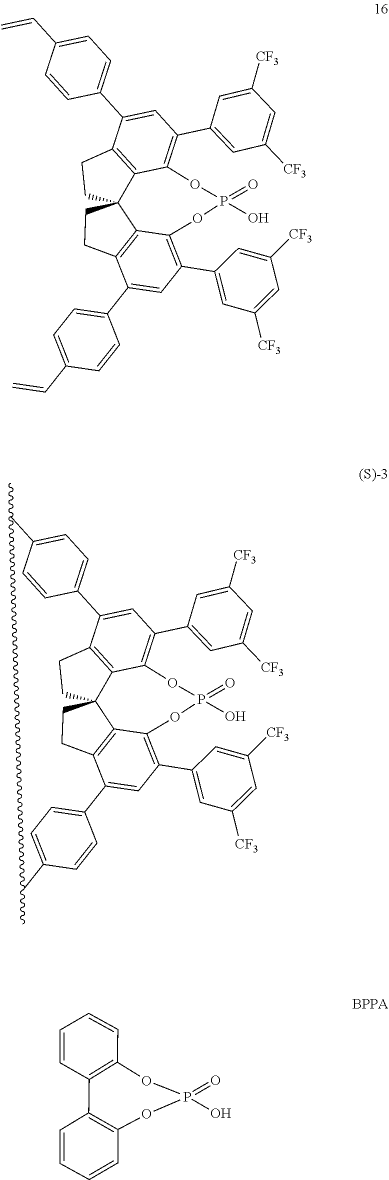

### Synthesis of C-11

Known diol C-10 (1.06 g, 1.57 mmol) was dissolved in dry DCM (30 mL) and the resulting solution was cooled to −50° C. To this solution was added a solution of Br₂ (500 mg, 3.13 mmol, 2 equiv.) in 5 mL of dry DCM under nitrogen while stirring. Upon addition, almost instant discoloration of the bromine solution was observed, and an orange color developed. The reaction mixture was stirred for an additional 10 minutes at −50° C., then warmed to room temperature. The resulting solution was washed with a 10% w/w aqueous solution of NaHSO₃ (15 mL) and with NaCl brine (15 mL), dried over anhydrous sodium sulfate, and filtered. The organic phase was then removed by rotary evaporation, and the remaining residue was directly purified by flash column chromatography on silica using 5% ethyl acetate in hexanes. C-11 (1.27 g, 1.52 mmol) was obtained as a beige solid in 97% yield.

R_{f}=0.65 (9:1 hexanes:ethyl acetate). IR (thin film, cm⁻¹): 3499, 1459, 1378, 1275, 1167, 1006, 902, 888, 846, 708, 682, 660. ¹H NMR (700 MHz, chloroform-d: δ=7.95-7.91 (m, 2H), 7.84 (s, 1H), 7.43 (s, 1H), 4.84 (s, 1H), 3.18 (ddd, J=17.0, 9.1, 1.4 Hz, 1H), 3.10 (ddd, J=16.9, 10.6, 7.8 Hz, 1H), 2.48 (ddd, J=13.2, 7.8, 1.3 Hz, 1H), 2.40-2.34 (m, 1H). ¹³C NMR (176 MHz, chloroform-d): δ=148.7, 146.5, 138.0, 133.7, 132.3, 132.1, 131.9, 131.7, 131.6, 129.4, 126.9, 125.5, 123.9, 122.4, 121.4, 120.8, 111.8, 60.8, 37.0, 32.7. ¹⁹F NMR (658 MHz, chloroform-d): δ=−62.9. HRMS (ESI+) (m/z): [M−H]⁻ calculated for C₃₃H₁₇Br₂F₁₂O₂⁻: 832.9389, found: 832.9379. [α]_{D}²⁵=−45.7° (c=1.40, CH₂Cl₂).

### Synthesis of C-12

To a solution of C-11 (1.32 mmol, 1.01 g), 4-vinylphenylboronic acid (975 mg, 6.59 mmol, 5 equiv.) and K₂CO₃ (912 mg, 6.59 mmol, 5 equiv.) in a degassed mixture of 1,4-dioxane/water (2:1, 20 mL), Pd(PPh₃)₄ (0.066 mmol, 5 mol %, 76 mg) in 5 mL of 1,4-dioxane was added using a syringe. The resulting reaction mixture was maintained at 70° C. with stirring for 5 hours. This mixture was subsequently cooled down to room temperature, acidified with 1M aqueous HCl, and concentrated under reduced pressure using rotary evaporation, with the aid of 2×50 mL of acetonitrile as an azeotroping solvent. The remaining residue was then directly purified by flash column chromatography on silica using 5% ethyl acetate in hexanes. C-12 was obtained as a white solid (1.030 g, 1.17 mmol) in 88% yield.

R_{f}=0.8 (9:1 hexanes:ethyl acetate); R_{f}=0.55 (95:5 hexanes:ethyl acetate). IR (thin film, cm⁻¹): 3499, 1392, 1373, 1330, 1275, 1167, 1131, 987, 893, 844, 707, 681. ¹H NMR (700 MHz, chloroform-d): δ=8.04 (d, J=1.6 Hz, 2H), 7.84 (s, 1H), 7.53 (d, J=8.2 Hz, 2H), 7.48-7.44 (m, 2H), 7.35 (s, 1H), 6.79 (dd, J=17.5, 10.9 Hz, 1H), 5.83 (d, J=17.6 Hz, 1H), 5.32 (d, J=10.9 Hz, 1H), 5.08 (s, 1H), 3.28 (ddd, J=16.5, 11.2, 7.6 Hz, 1H), 3.17 (dd, J=16.6, 8.5 Hz, 1H), 2.56 (dd, J=13.1, 7.5 Hz, 1H), 2.39 (td, J=12.3, 8.9 Hz, 1H). ¹³C NMR (176 MHz, chloroform-d): δ=149.1, 144.4, 139.4, 139.1, 136.6, 136.4, 136.3, 132.4, 131.9, 131.8, 131.7, 131.5, 131.3, 130.8, 129.5, 129.5, 129.3, 128.6, 126.8, 126.4, 126.3, 125.7, 125.4, 124.1, 122.6, 121.0, 120.9, 120.9, 120.9, 120.9, 114.2, 113.9, 58.4, 37.4, 31.2. ¹⁹F NMR (658 MHz, chloroform-d): δ=−62.8. HRMS (ESI+) (m/z): [M−H]⁻ calculated for C₄₉H₃₁F₁₂O₂⁻, 879.2138, found 879.2114. [α]_{D}²⁵=−13.7° (c=2.70, CH₂Cl₂).

### Synthesis of 16

To a stirred solution of C-12 (900 mg, 1.02 mmol) in pyridine (4.0 mL, 51.1 mmol, 4.00 equiv.) was added POCl₃ (478 μL, 5.11 mmol, 5.00 equiv.). The mixture was stirred at 70° C. for 12 h, then added to a flask charged with water (40 mL) and stirred at 100° C. for 12 h. After cooling to room temperature, the mixture was diluted with DCM (40 mL). The organic layer was separated and the aqueous layer was extracted with DCM (3×40 mL). The organic layer was then washed with 6 N aqueous HCl (3×40 mL). The combined organic layers were then dried with brine and anhydrous sodium sulfate and concentrated under reduces pressure to obtain a pale yellow solid. The crude product was then purified by column using 2% methanol in DCM. The obtained product was then re-dissolved in DCM, washed with 6 N aqueous HCl, dried over anhydrous sodium sulfate, and concentrated under reduced pressure to obtain the free acid form of 16 (732 mg, 0.77 mmol) in 76% yield.

IR (thin film, cm⁻¹): 2946, 1629, 1513, 1479, 1390, 1371, 1336, 1276, 1164, 1125, 1087, 1055, 1008, 891, 840, 755, 731, 681, 646. ¹H NMR (700 MHz, chloroform-d): δ=7.95 (s, 1H), 7.72 (ddd, J=21.0, 8.4, 2.0 Hz, 3H), 7.38 (d, J=2.0 Hz, 0H), 6.98 (ddd, J=17.7, 11.0, 2.0 Hz, 1H), 6.03 (dd, J=17.7, 2.0 Hz, 1H), 5.51 (dd, J=11.0, 1.9 Hz, 1H), 3.48 (ddd, J=17.3, 11.1, 6.6 Hz, 1H), 3.19 (dd, J=16.6, 7.7 Hz, 1H), 2.67 (dd, J=12.5, 6.4 Hz, 1H), 2.46 (q, J=11.0 Hz, 1H). ¹³C NMR (176 MHz, chloroform-d): δ=144.4, 140.9, 139.4, 138.7, 137.0, 136.3, 132.7, 131.3, 131.1, 129.5, 128.8, 126.4, 125.6, 124.1, 122.5, 121.0, 120.7, 114.4, 60.5, 38.4, 30.5. ¹⁹F NMR (658 MHz, chloroform-d): δ=−62.60. ³¹P NMR (283 MHz, chloroform-d): δ=−7.66. HRMS (ESI+) (m/z): [M−H]⁻ calculated for C₄₉H₃₀F₁₂O₄P, 946.1696, found 946.1678. [α]_{D}²⁵=−162.5° (c=1.45, CH₂Cl₂).

### Preparation of Immobilized Catalyst (S)-SPINOL-PS ((S)-3)

Solution A was prepared by adding polyvinyl alcohol (PVA, Mw 89,000-98,000) (39 mg) in water (39 mL) and heating at 80° C. with stirring for 60 minutes to dissolve the PVA. The resulting solution was cooled to room temperature and degassed by sparging with nitrogen for 15 minutes.

Solution B was prepared by mixing styrene (2.73 g), divinylbenzene (80%, 94 mg) (both of which had been washed with 3N aqueous NaOH solution, passed through an alumina plug, and dried over sodium sulfate), 16 (900 mg) and toluene (1.25 mL).

Solution A and Solution B were mixed in a 75 mL cylindrical-shaped vessel with a PTFE screw cap (Chem Glass CG-1880-10), and dibenzoyl peroxide (75%, water stabilized) (42 mg) was added. The mixture was degassed by sparging with nitrogen for 5 minutes at room temperature and heated to 95° C. while stirring at 1100 rpm. After 36 hours, the reaction mixture was cooled to room temperature, and the resulting solid material was collected by vacuum filtration. The crude product was sequentially washed with water, methanol, THF, DCM, and hexane and dried in high vacuum for 12 hours to result in 3.5 g of white spherical particles of (S)-SPINOL-PS ((S)-3).

The solvents used to wash the crude reaction product were combined and evaporated to dryness. This produced 0.25 g of nonvolatile residue which contained only trace quantities of the starting monomeric acid 16, as determined by ¹⁹F NMR. The residue appeared to be mostly a mixture of organic-soluble styrene-divinylbenzene oligomers, as determined by ¹H NMR.

Elemental analysis of (S)-3: C=88.24%, H=7.72%, F=2.56%. ¹⁹F NMR (471 MHz, benzene-d₆): −62.3 ppm. FTIR (ATR; cm⁻¹): 3059, 3024, 2921, 2849, 1601, 1492, 1451, 1372, 1279, 1179, 1133, 1026, 1010, 903, 837, 755, 695.

### Example 2a: Protecting Group Reactions Using Immobilized Catalyst (R)-2

Tables 1 and 2 list yields and regioselectivity ratios (rr) for MOC and MOP protection reactions of various 2,3-diols carried out according to the procedures described above using immobilized catalyst (R)-2 and unsupported catalyst (R)-1. Table 3 lists yield and C4:C3 rr for MOP protection of a 3,4-diol, L-rhamnal (4t). 2-Nap denotes 2-naphthyl.

TABLE 1

| Ex. | Diol | Protected Diol | Yield | C2:C3 rr |
|---|---|---|---|---|
| 1A | 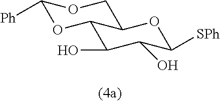 | 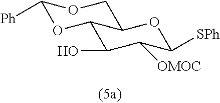 | (R)-2: 95% (R)-1: 95% | (R)-2: >25:1 rr (R)-1: >25:1 rr |
| 1B | 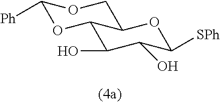 | 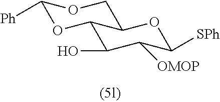 | (R)-2: 85% (R)-1: 72% | (R)-2: >25:1 rr (R)-1: >25:1 rr |
| 1C | 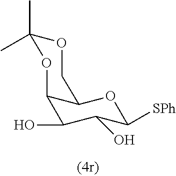 | 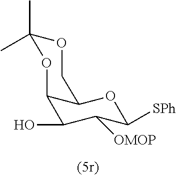 | (R)-2: 81% (R)-1: 84% | (R)-2: 21:1 rr (R)-1: 20:1 rr |
| 1D | 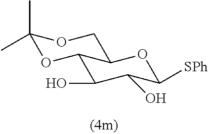 | 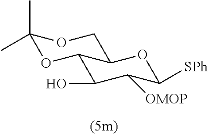 | (R)-2: 89% (R)-1: 98% | (R)-2: 19:1 rr (R)-1: 12:1 rr |
| 1E | 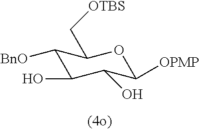 | 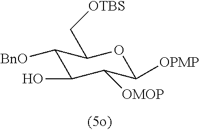 | (R)-2: 95% (R)-1: 88% | (R)-2: >25:1 rr (R)-1 7:1 rr |
| 1F | 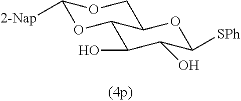 | 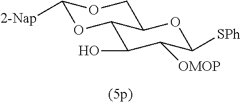 | (R)-2: 89% (R)-1: 92% | (R)-2: >25:1 (R)-1: 3:1 rr |
| 1G | 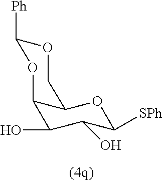 | 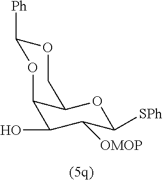 | (R)-2: 78% (R)-1: 93% | (R)-2: 15:1 rr (R)-1: 20:1 rr |

TABLE 2

| Ex. | Diol | Protected Diol | Yield | C3:C2 rr |
|---|---|---|---|---|
| 1H | 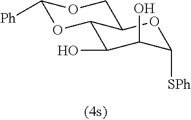 | 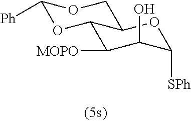 | (R)-2: 67% (R)-1: 60% | (R)-2: 16:1 rr (R)-1: 13:1 rr |
| 1J | 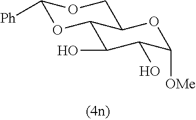 [SMILES: COC1OC2COC(c3ccccc3)OC2C(O)C1O] | 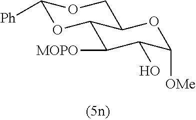 | (R)-2: 73% (R)-1: 69% | (R)-2: 1.1:1 rr (R)-1: 6.3:1 rr |

TABLE 3

| | | | | C4:C3 |
|---|---|---|---|---|
| Ex. | Diol | Protected Diol | Yield | rr |
| 1K | 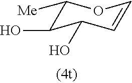 | 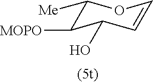 | (R)-2: 92% BPPA: 93% | (R)-2: 8.2:1 rr BPPA: 1.1 rr |

Immobilized catalyst (R)-2 provided selective protection at the C2 position for almost every diol studied. In several instances (5m, 5o, and 5p), the immobilized catalyst provided significantly higher regioselectivity than that provided the corresponding unsupported catalyst (R)-1. In the case of the 3,4-diol 4t (L-rhamnal), MOP protection using immobilized catalyst (R)-2 resulted in formation of the MOP acetal at the more sterically hindered C4 position with 8.2:1 selectivity, while using a non-chiral phosphoric acid derived from 2,2′-biphenol (BPPA) resulted in a 1:1 mixture of the C3 and C4 isomers.

### Example 2b: Protecting Group Reactions Using Immobilized Catalyst 3

Table 4 lists yields and C3:C2 regioselectivity ratios for MOC and MOP protection reactions carried out on various diols using immobilized catalyst 3 and unsupported catalyst 16. Examples 2A-2G show results of reactions using (S)-3 and (S)-16; Examples 2H and 2J show results of reactions using (R)-3 and (R)-16.

TABLE 4

| Ex. | Diol | Protected Diol | Yield | C3:C3 rr |
|---|---|---|---|---|
| 2A | 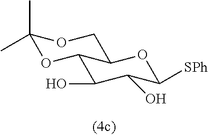 | 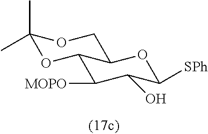 | (S)-3: 88% (S)-16: 83% | (S)-3: 6.1 rr (S)-16: 3:1 rr |
| 2B | 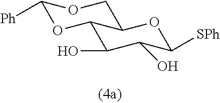 | 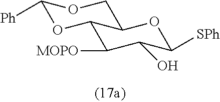 | (S)-3: 78% (S)-16: 75% | (S)-3: 8:1 rr (S)-16: 5:1 rr |
| 2C | 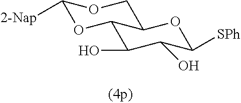 | 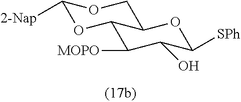 | (S)-3: 91% (S)-16: 93% | (S)-3: 10:1 rr (S)-16: 6:1 rr |
| 2D | 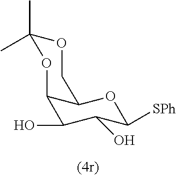 | 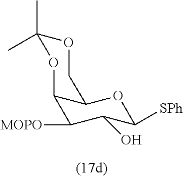 | (S)-3: 85% (S)-16: 74% | (S)-3: 5:1 rr (S)-16: 10:1 rr |
| 2E | 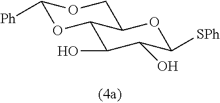 | 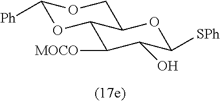 | (S)-3: 80% (S)-16: 87% | (S)-3: 9:1 rr (S)-16: 6.1 rr |
| 2F | 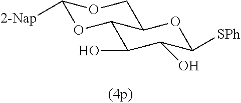 | 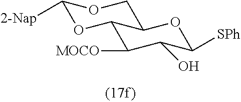 | (S)-3: 80% (S)-16: 82% | (S)-3: 14:1 rr (S)-16: 11:1 rr |
| 2G | 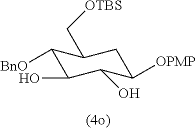 | 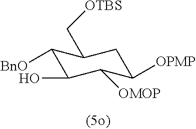 | (S)-3: 91% (S)-16: 87% | (S)-3: >25:1 rr (S)-16: 5:1 rr |
| 2H | 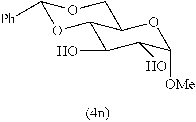 [SMILES: COC1OC2COC(c3ccccc3)OC2C(O)C1O] | 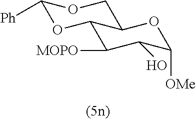 | (R)-3: 96% (R)-16: 55% | (R)-3: 3:1 rr (R)-16: 1:3 rr |
| 2J | 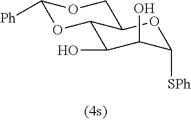 | 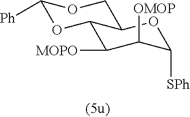 | (R)-3%: 58% (R)-16: 78% | 5u:5s rr (R)-3: 2.2:1 (R)-16: 4:1 |
| | | 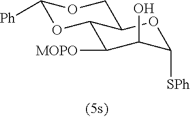 | | |

The immobilized catalyst (S)-3 generally provided higher C3:C2 selectivity than that provided by the corresponding unsupported catalyst (S)-16.

In Example 2H, the immobilized and unsupported catalysts gave selective protection of different positions. MOP protection of diol 4n using the immobilized catalyst (R)-3 selectively protected the C3 position (3:1 rr), while MOP protection of diol 4n using the unsupported catalyst (R)-16 selectively protected the C2 position (1:3 rr).

In Example 2J, formation of diol with MOP protection at both the C2 and C3 positions (5u) and diol with MOP protection only at the C3 position (5s) was observed. As noted in the table, the regioselectivity ratio for this reaction describes the selectivity of formation of the dual-protected diol 5u.

### Example 3a: Recyclability of Immobilized Catalyst (R)-2

To demonstrate recyclability of the immobilized catalysts, the protecting group reaction shown in Example 1B was carried out ten times in succession using a single sample of immobilized catalyst (R)-2. For the first nine reaction cycles, 1.0 equiv. of diol 4a (which had been pre-dried by the azeotropic removal of moisture with toluene), activated 4 Å molecular sieves, 0.5 mol % (based on diol) of immobilized catalyst (R)-2, and anhydrous DCM were charged to a reaction vessel. The mixture was cooled to about −78° C., 1.2 equiv. of 2-MOP was added, and the reaction mixture was mixed for 18 hours, after which the reaction was complete as determined by TLC. The reaction mixture was quenched with 5% triethylamine in methanol and filtered to recover the catalyst. The filtrate was dried under reduced pressure to obtain the reaction product as a white solid. The catalyst was partitioned in DCM to separate it from the molecular sieves; the catalyst floated in DCM while the molecular sieves settled in DCM. The recovered catalyst was collected and washed successively with 6N HCl, 1N HCl, water, acetone, DCM, and hexane. The washed catalyst was dried under reduced pressure and re-used for the next reaction.

For the tenth reaction cycle, the recycled catalyst sample from reaction cycles 1-9 was used with 5.0 equiv. of diol 4a, instead of 1.0 equiv. as in the preceding samples. Thus, the catalyst loading for the tenth reaction cycle was 0.1 mol % based on diol, instead of 0.5 mol %. Reaction time for the tenth cycle was 36 hours.

Reaction conditions and catalyst performance (yield and C2 rr) for each reaction cycle are summarized in Table 5. High conversion and regioselectivity were maintained throughout all cycles, including the final cycle, which utilized just 20% of the catalyst loading used for the previous cycles. Notably, the yield and selectivity of the catalyst after ten cycles were essentially equivalent to those achieved in the first cycle.

TABLE 5

| | Catalyst loading | | | |
|---|---|---|---|---|
| Cycle | (mol % on diol) | Reaction time | Yield | C2:C3 rr |
| 1 | 0.5 | 18 hr | 99% | >25:1 |
| 2 | 0.5 | 18 hr | 95% | 23:1 |
| 3 | 0.5 | 18 hr | 97% | >25:1 |
| 4 | 0.5 | 18 hr | 99% | >25:1 |
| 5 | 0.5 | 18 hr | 97% | >25:1 |
| 6 | 0.5 | 18 hr | 99% | >25:1 |
| 7 | 0.5 | 18 hr | 99% | >25:1 |
| 8 | 0.5 | 18 hr | 99% | >25:1 |
| 9 | 0.5 | 18 hr | 99% | >25:1 |
| 10 | 0.1 | 36 hr | 99% | >25:1 |

### Example 3b: Recyclability of Immobilized Catalyst (S)-3

Recyclability of immobilized catalyst (S)-3 was demonstrated by carrying out the protecting group reaction shown in Example 2C seven times in succession using a single sample of catalyst. 1.0 equiv. of diol 4p (which had been pre-dried by the azeotropic removal of moisture with toluene), activated 4 Å molecular sieves, 2.0 mol % (based on diol) of immobilized catalyst (S)-3, and anhydrous DCM were charged to a reaction vessel. The mixture was cooled to about −78° C., 1.2 equiv. of 2-MOP was added, and the reaction mixture was mixed for 24 hours, after which the reaction was complete as determined by TLC. The reaction mixture was quenched with 0.5 M ammonia in methanol and filtered to recover the catalyst. The filtrate was dried under reduced pressure to obtain the crude reaction product, and the recovered catalyst was partitioned in DCM to separate it from the molecular sieves. The catalyst was collected, washed with DCM, dried under reduced pressure, and re-used for the next reaction. Following completion of cycle 6 and before being used for cycle 7, the catalyst was washed with 6N HCl prior to washing with DCM.

Reaction conditions and catalyst performance (yield and C3:C2 rr) for each reaction cycle are shown in Table 6. High conversion and regioselectivity were maintained throughout all reaction cycles. Though regioselectivity decreased markedly during cycle 6, the performance of the catalyst sample for cycle 7, after being washed with 6N HCl following cycle 6, was substantially similar to its performance in the initial reaction cycle.

TABLE 6

| | Catalyst loading | | | |
|---|---|---|---|---|
| Cycle | (mol % on diol) | Reaction time | Yield | C3:C2 rr |
| 1 | 2.0 | 24 hr | 88% | 11.2 |
| 2 | 2.0 | 24 hr | 95% | 10.9 |
| 3 | 2.0 | 24 hr | 95% | 10.6 |
| 4 | 2.0 | 24 hr | 95% | 9.6 |
| 5 | 2.0 | 24 hr | 92% | 9.5 |
| 6 | 2.0 | 24 hr | 94% | 6.4 |
| 7 | 2.0 | 24 hr | 93% | 10.3 |

1. A compound of Formula (I) 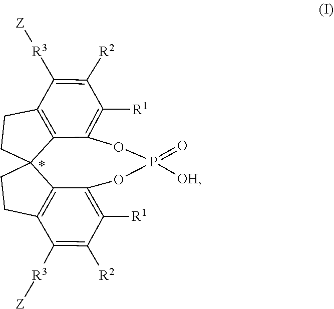
wherein each R¹ is independently selected from
H;
halogen;
cyano;
nitro;
C₁₋₆ alkyl, unsubstituted or substituted with one or more substituents selected from halo, OH, substituted or unsubstituted aryl, substituted or unsubstituted heteroaryl, NR⁵R⁶, and C(═O)R⁷, wherein R⁷ is selected from OH, O(C₁₋₆ alkyl), and C₁₋₆ alkyl;
C₃₋₆ cycloalkyl, unsubstituted or substituted with one or more substituents selected from halo, OH, substituted or unsubstituted aryl, and substituted or unsubstituted heteroaryl;
aryl or heteroaryl, unsubstituted or substituted with one or more R⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R⁹, and NR¹⁰R¹¹;
C(═O)R¹²;
NR¹³R¹⁴;
OR¹⁷, wherein R¹⁷ is selected from H, C₁₋₆ alkyl, C₁₋₆ haloalkyl, C₃₋₆ cycloalkyl, substituted or unsubstituted aryl, and substituted or unsubstituted heteroaryl; and
silyl, unsubstituted or substituted with one, two, or three substituents independently selected from aryl and C₁₋₆ alkyl;
wherein each R⁵, R⁶, R¹⁰, R¹¹, R¹³, and R¹⁴ is independently selected from H, unsubstituted C₁₋₆ alkyl, and C₁₋₆ alkyl substituted with one or more substituents selected from OH, halo, NH₂, aryl, and heteroaryl;
wherein each R⁷, R⁹, and R¹² is independently selected from OH, unsubstituted or substituted O(C₁₋₆ alkyl), unsubstituted or substituted C₁₋₆ alkyl, and unsubstituted or substituted NH₂;
each R² is independently selected from H, OH, halo, NH₂, C₁₋₆ alkyl, C₁₋₆ haloalkyl, substituted or unsubstituted aryl, and substituted or unsubstituted heteroaryl;
each R³ is independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing; and
Z denotes a solid support,
wherein * denotes a center of chirality.

2. The compound of claim 1, wherein the center of chirality is in an (S) conformation.

3. The compound of claim 1, wherein each R¹ is 3,5-bis(trifluoromethyl)phenyl and each R² is H.

4. The compound of claim 1, wherein at least one R¹ is H.

5.-9. (canceled)

10. The compound of claim 1, wherein at least one R¹ is C₁₋₆ alkyl, unsubstituted or substituted with one or more substituents selected from halo, OH, substituted or unsubstituted aryl, substituted or unsubstituted heteroaryl, NR₅R₆, and C(═O)R⁷.

11. (canceled)

12. The compound of claim 1, wherein at least one R¹ is C₃₋₆ cycloalkyl, unsubstituted or substituted with one or more substituents selected from halo, OH, substituted or unsubstituted aryl, and substituted or unsubstituted heteroaryl.

13. (canceled)

14. The compound of claim 1, wherein at least one R¹ is aryl or heteroaryl, unsubstituted or substituted with one or more R₈ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R⁹, and NR¹⁰R¹¹.

15.-21. (canceled)

22. The compound of claim 1, wherein at least one R² is H.

23. (canceled)

24. The compound of claim 1, wherein at least one R² is OH.

25. (canceled)

26. The compound of claim 1, wherein at least one R² is C₁₋₆ alkyl.

27. (canceled)

28. The compound of claim 1, wherein at least one R² is substituted or unsubstituted aryl.

29. (canceled)

30. The compound of claim 1, wherein both R³ are selected from C₁₋₆ alkylene, phenylene, alkylenephenylene, and combinations of the foregoing.

31. The compound of claim 30, wherein both R³ are 4-alkylenephenylene.

32. The compound of claim 1, wherein Z is selected from polymer beads, clays, zeolites, and silicon-based resins.

33. (canceled)

34. (canceled)

35. The compound of claim 32, wherein Z is a polymer bead and the polymer bead comprises polystyrene.

36. The compound of claim 32, wherein Z is a polymer bead and the polymer bead is cross-linked.

37. The compound of claim 36, wherein the cross-linked polymer bead comprises poly(styrene-co-divinylbenzene).

38. A method of forming a polyol having a protected hydroxyl group, comprising mixing a polyol comprising two or more hydroxyl groups, a protecting group compound, a compound according to claim 1, and a solvent to form a product comprising a polyol having a protected hydroxyl group,
wherein the solvent is a solvent for the polyol comprising two or more hydroxyl groups and the protecting group compound.

39.-53. (canceled)

54. A method of protecting multiple hydroxyl groups of a polyol, the method comprising mixing
a polyol comprising two or more hydroxyl groups;
two or more protecting group compounds;
a compound according to claim 1;
a compound according to Formula (II) 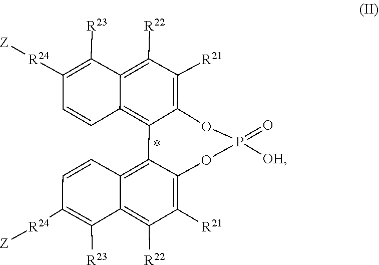
wherein each R²¹ is independently selected from
H,
cyano,
nitro,
C₁₋₆ alkyl, unsubstituted or substituted with one or more substituents selected from halo, OH, substituted or unsubstituted aryl, substituted or unsubstituted heteroaryl, NR²⁵R²⁶, and C(═O)R²⁷,
C₃₋₆ cycloalkyl, unsubstituted or substituted with one or more substituents selected from halo, OH, substituted or unsubstituted aryl, and substituted or unsubstituted heteroaryl,
aryl or heteroaryl, unsubstituted or substituted with one or more R²⁸ groups independently selected from halo, C₁₋₆ alkyl, C₁₋₆ haloalkyl, C₃₋₆ cycloalkyl, 1-adamantyl, 2-adamantyl, substituted or unsubstituted aryl, substituted or unsubstituted heteroaryl, OH, O(C₁₋₆ alkyl), O(C₁₋₆ haloalkyl), O(C₃₋₆ cycloalkyl), O(aryl), O(heteroaryl), cyano, nitro, C(═O)R²⁹, and NR³⁰R³¹,
C(═O)R³²,
NR³³R³⁴, and
OR³⁵, wherein R³⁵ is selected from H, substituted or unsubstituted C₁₋₆ alkyl, substituted or unsubstituted C₃₋₆ cycloalkyl, substituted or unsubstituted aryl, and substituted or unsubstituted heteroaryl,
wherein each R²⁵, R²⁶, R³⁰, R³¹, R³³, and R³⁴ is independently selected from H, unsubstituted C₁₋₆ alkyl, and C₁₋₆ alkyl substituted with one or more substituents selected from OH, halo, NH₂, aryl, and heteroaryl,
wherein each R²⁷, R²⁹, and R³² is independently selected from OH, unsubstituted or substituted O(C₁₋₆ alkyl), unsubstituted or substituted C₁₋₆ alkyl, and unsubstituted or substituted NH₂,
each R²² and R²³ is independently selected from H, OH, halo, NH₂, C₁₋₆ alkyl, C₁₋₆ haloalkyl, substituted or unsubstituted aryl, and substituted or unsubstituted heteroaryl;
each R²⁴ is independently selected from C₁₋₆ alkylene, —SiH₂—, —SiH(alkyl)—, —Si(dialkyl)—, substituted or unsubstituted arylene, substituted or unsubstituted heteroarylene, and combinations of the foregoing,
Z denotes a solid support,
wherein * denotes an axis of chirality; and
a solvent to form a product comprising a polyol having multiple protected hydroxyl groups,
wherein the solvent is a solvent for the polyol comprising two or more hydroxyl groups and the two or more protecting group compounds.

55.-75. (canceled)

76. The compound of claim 1, wherein each R¹ is 3,5-bis(trifluoromethyl)phenyl, each R² is H, each R³ is 4-alkylenephenylene, and Z is a polymer bead comprising cross-linked polystyrene.
